(19) United States
(12) Patent Application Publication
(10) Pub. No.: US 2023/0176054 A1
(43) Pub. Date: Jun. 8, 2023

(54) CORONAVIRUS ASSAY

(71) Applicant: LumiraDx UK Ltd., London (GB)

(72) Inventor: Victor Manneh, Encinitas, CA (US)

(21) Appl. No.: 17/913,037

(22) PCT Filed: Mar. 22, 2021
(86) PCT No.: PCT/US2021/023532

§ 371 Date: Sep. 20, 2022

Related U.S. Application Data
(60) Provisional application No. 63/032,378, filed on May 29, 2020.
(60) Provisional application No. 63/009,906, filed on Apr. 14, 2020.
(60) Provisional application No. 62/992,681, filed on Mar. 20, 2020.

(51) Int. Cl. G01N 33/569; G01N 33/53

(52) U.S. Cl. CPC G01N 33/56983; G01N 33/5304; G01N 2333/165; G01N 2470/04

(57) ABSTRACT

Disclosed herein are methods of detecting a coronavirus, e.g., SARS-CoV-2, using a sandwich assay.

### RELATED APPLICATIONS

The present application claims the benefit of and priority to U.S. Provisional Patent Application No. 62/992,681, filed Mar. 20, 2020; U.S. Provisional Patent Application No. 63/009,906, filed Apr. 14, 2020; and U.S. Provisional Patent Application No. 63/032,378, filed May 29, 2020, the entire disclosure of each of which is incorporated herein by reference in its entirety.

### FIELD OF THE INVENTION

Disclosed herein are methods of detecting a coronavirus, e.g., SARS-CoV-2, using a binding assay.

### BACKGROUND

Angiotensin-converting enzyme-2 (ACE2) is a functional receptor for severe acute respiratory syndrome (SARS) coronaviruses and human coronavirus NL63. During the SARS-CoV epidemic of 2002-2005, ACE2 was found to be a receptor for SARS-CoV (i.e., SARS-CoV-1) (Li et al., (2003) NATURE 426(6965):450-4). ACE2 also is the receptor for SARS-CoV-2 (Zhou et al., (2020) NATURE 579(7798):270-273). In addition, ACE2 is used as a receptor by the human coronavirus NL63 (HCoV-NL63) (Hofmann et al., (2005) PROC NATL ACAD SCI USA 102(22):7988-93).

There is currently a worldwide public health emergency caused by the SARS-CoV-2 pandemic. In some cases, viral infection is asymptomatic, leading to a high risk for silent spread of the virus. Identification and isolation of subjects infected by the SARS-CoV-2 (and other ACE2-binding viruses) is important for reducing its spread. Methods of testing for viral infection exist but can be time-consuming and/or inaccurate. Accordingly, there is a need in the art for improved methods of detecting a virus (e.g., a coronavirus) in a subject.

### SUMMARY OF THE INVENTION

The invention is based, in part, on the discovery that a soluble ACE2 receptor is capable of functioning as a high-affinity binding moiety suitable for use in a viral detection assay, such as a sandwich assay, as described in further detail herein. It has been further discovered that the addition of salt (e.g., NaCl) to a sample buffer (1) increases recovery of viral protein from a sample and (2) and prevents prevent loss of viral protein on the surface of a microfluidic device, thereby improving the performance of a viral detection assay.

Accordingly, the disclosure relates in part to a method for detecting a coronavirus in a sample from a subject. The method includes subjecting the sample to a double binding moiety sandwich assay comprising a first and a second binding moiety, wherein the first binding moiety comprises a soluble ACE2 receptor, or a variant or fragment thereof, or an anti-coronavirus antibody. The first binding moiety is labeled with a detectable label or a capture agent, and the second binding moiety is attached to a detectable label or a capture agent. The first binding moiety and the second binding moiety can bind the coronavirus to form a complex comprising the first binding moiety, the coronavirus, and the second binding moiety.

In certain embodiments, at least one of the first binding moiety and the second binding moiety comprises a soluble ACE2 receptor, or a variant or fragment thereof. In certain embodiments, the first binding moiety comprises a soluble ACE2 receptor and the second binding moiety comprises an anti-coronavirus antibody. In certain embodiments, the first binding moiety comprises an anti-coronavirus antibody and the second binding moiety comprises a soluble ACE2 receptor. In certain embodiments, the soluble ACE2 receptor comprises an ACE2-Fc fusion protein.

In certain embodiments, the first binding moiety comprises an anti-coronavirus antibody. In certain embodiments, the second binding moiety comprises an anti-coronavirus antibody.

In certain embodiments, the anti-coronavirus antibody or fragment thereof binds to a spike protein, a nucleocapsid protein, an envelope protein, a membrane protein, or a hemagglutinin-esterase dimer protein of a coronavirus.

In certain embodiments, the first binding moiety is associated, e.g., covalently associated, with the detectable label. In certain embodiments, the first binding moiety is associated, e.g., covalently associated, with the capture agent. In certain embodiments, the second binding moiety is associated, e.g., covalently associated, with the detectable label. In certain embodiments, the second binding moiety is associated, e.g., covalently associated, with the capture agent. In certain embodiments, the detectable label comprises a fluorescent label, e.g., a fluorescent latex bead. In certain embodiments, the capture agent comprises a magnetic bead.

In certain embodiments, the method is performed within a microfluidic device, such as that described in International Patent Application No. PCT/US2021/013325, which is incorporated by reference herein in its entirety.

In certain embodiments, the coronavirus is SARS-CoV-2.

In certain embodiments, the sample comprises blood, serum, or plasma.

In certain embodiments, the sample is contacted with an exonuclease prior to subjecting the sample to the double binding moiety sandwich assay. In certain embodiments, the exonuclease is present in an amount of about 0.002 to about 1 U/mL. In certain embodiments, the exonuclease is benzonase.

In certain embodiments, the sample is contacted with a latex particle prior to subjecting the sample to the double binding moiety sandwich assay.

In certain embodiments, the sample is contacted with a buffer comprising a salt solution prior to subjecting the sample to the double binding moiety sandwich assay.

In certain embodiments, upon subjecting the sample to the double binding moiety sandwich assay, the presence of the coronavirus is detected.

In another aspect, the disclosure relates to a microfluidic device for detecting a coronavirus in a sample from a subject. The device includes a microchannel comprising a first and a second binding moiety dried within, wherein the first binding moiety comprises a soluble ACE2 receptor, or a variant or fragment thereof, or an anti-coronavirus antibody. The first binding moiety is labeled with a detectable label or a capture agent and the second binding moiety is attached to a detectable label or a capture agent, wherein the first and second binding moieties, when solubilized with the sample, form a complex comprising the first binding moiety, the coronavirus, and the second binding moiety.

In certain embodiments, at least one of the first binding moiety and the second binding moiety comprises a soluble ACE2 receptor, or a variant or fragment thereof. In certain embodiments, the first binding moiety comprises a soluble ACE2 receptor and the second binding moiety comprises an anti-coronavirus antibody. In certain embodiments, the first binding moiety comprises an anti-coronavirus antibody and the second binding moiety comprises a soluble ACE2 receptor. In certain embodiments, the soluble ACE2 receptor comprises an ACE2-Fc fusion protein.

In certain embodiments, the second binding moiety comprises a soluble ACE2 receptor, or a variant or fragment thereof. In certain embodiments, the second binding moiety comprises an anti-coronavirus antibody or fragment thereof, wherein the fragment binds to the coronavirus.

In certain embodiments, the anti-coronavirus antibody binds to a spike protein, a nucleocapsid protein, an envelope protein, a membrane protein, or a hemagglutinin-esterase dimer protein of a coronavirus. In certain embodiments, the anti-coronavirus antibody or fragment thereof binds to a nucleocapsid protein.

In certain embodiments, the microfluidic device comprises a microchannel comprising an exonuclease dried within. In certain embodiments, the first binding moiety is associated with, e.g., covalently associated, with the detectable label. In certain embodiments, the first binding moiety is associated, e.g., covalently associated, with the capture agent. In certain embodiments, the second binding moiety is associated, e.g., covalently associated, with the detectable label. In certain embodiments, the second binding moiety is associated, e.g., covalently associated, with the capture agent. In certain embodiments, the detectable label comprises a fluorescent label, e.g., a fluorescent latex bead. In certain embodiments, the capture agent comprises a magnetic bead. In certain embodiments, coronavirus is SARS-CoV-2. In certain embodiments, the sample comprises blood, serum, or plasma.

In another aspect, the disclosure relates to a method for detecting an anti-coronavirus spike protein antibody in a sample from a subject. The method comprises subjecting the sample to a double binding moiety sandwich assay comprising a first and a second binding moiety, wherein the first binding moiety comprises an S1 or an S2 subunit of a coronavirus spike protein, or a fragment thereof, associated with a detectable label or capture agent, and wherein the second binding moiety is associated with a detectable label or capture agent, and wherein the first binding moiety and the second binding moiety can bind the anti-coronavirus spike protein antibody to form a complex comprising the first binding moiety, the anti-coronavirus spike protein antibody, and the second binding moiety, whereupon the formation of the complex is indicative of the presence of the anti-coronavirus spike protein antibody in the sample.

In certain embodiments, the second binding moiety comprises the S1 or the S2 subunit of the coronavirus spike protein, or a fragment thereof. In certain embodiments, the first and/or second binding moiety comprises the S1 and S2 subunits of the coronavirus spike protein, or a fragment thereof.

In certain embodiments, the second binding moiety comprises an anti-IgG, and anti-IgA, or an anti-IgM antibody, or a fragment of any of the foregoing.

In certain embodiments, the first binding moiety is associated, e.g., covalently associated, with the detectable label. In certain embodiments, the first binding moiety is associated, e.g., covalently associated, with the capture agent.

In certain embodiments, the second binding moiety is associated, e.g., covalently associated, with the detectable label. In certain embodiments, the second binding moiety is associated, e.g., covalently associated, with the capture agent.

In certain embodiments, the detectable label comprises a fluorescent particle, e.g., a fluorescent latex bead.

In certain embodiments, the capture agent comprises a magnetic bead.

In certain embodiments, the method is performed within a microfluidic device.

In certain embodiments, the coronavirus is SARS-CoV-2.

In certain embodiments, the sample comprises blood, serum, or plasma.

In certain embodiments, the sample is contacted with a latex particle prior to subjecting the sample to the double binding moiety sandwich assay.

In certain embodiments, the sample is contacted with a buffer comprising a salt solution prior to subjecting the sample to the double binding moiety sandwich assay.

In certain embodiments, upon subjecting the sample to the double binding moiety sandwich assay, the presence of the anti-coronavirus spike protein antibody is detected.

In another aspect, the disclosure relates to a microfluidic device for detecting an anti-coronavirus spike protein antibody in a sample from a subject, the device comprising a microchannel comprising a first and a second binding moiety dried within, wherein the first binding moiety comprises an S1 or an S2 subunit of a coronavirus spike protein, or a fragment thereof, and is labeled with a detectable label or a capture agent, and wherein the second binding moiety is attached to a detectable label or a capture agent, and wherein the first and second binding moieties, when solubilized with the sample, form a complex comprising the first binding moiety, the anti-coronavirus spike protein antibody, and the second binding moiety.

In certain embodiments, the second binding moiety comprises the S1 or the S2 subunit of the coronavirus spike protein, or a fragment thereof. In certain embodiments, the first and/or second binding moiety comprises the S1 and S2 subunits of the coronavirus spike protein, or a fragment thereof.

In certain embodiments, the second binding moiety comprises an anti-IgG, and anti-IgA, or an anti-IgM antibody, or a fragment of any of the foregoing.

In certain embodiments, the first binding moiety is associated, e.g., covalently associated, with the detectable label. In certain embodiments, the first binding moiety is associated, e.g., covalently associated, with the capture agent.

In certain embodiments, the second binding moiety is associated, e.g., covalently associated, with the detectable label. In certain embodiments, the second binding moiety is associated, e.g., covalently associated, with the capture agent.

In certain embodiments, the detectable label comprises a fluorescent particle, e.g., a fluorescent latex bead.

In certain embodiments, the capture agent comprises a magnetic bead.

In certain embodiments, the coronavirus is SARS-CoV-2.

In certain embodiments, the sample comprises blood, serum, or plasma.

### BRIEF DESCRIPTION OF THE DRAWINGS

In order to understand the invention and to demonstrate how it may be carried out in practice, embodiments are now described, by way of non-limiting example only, with reference to the accompanying drawings in which:

FIG. 1 is a schematic diagram demonstrating assay 1. In assay 1, magnetic beads (mags) are coupled to anti-human-IgMs (or IgGs, not shown) to capture IgMs (or IgGs) present in a sample. A subpopulation of those IgMs that are capable of binding to Spike (S), a SARS-CoV-2 surface protein can be detected by the presence of the fluorescently-labeled latex bead to which the S protein is attached.

FIG. 2 is a schematic diagram demonstrating assay 2. In assay 2, a SARS-CoV-2 Spike protein is biotinylated and a second SARS-CoV-2 Spike protein is conjugated to fluorescent latex, resulting in a fluorescent, bridged complex when contacted with an infected sample.

FIG. 3 is a schematic diagram demonstrating assay 3. In assay 3, human angiotensin-converting enzyme 2 (ACE2), modified with an Fc domain, captures fluorescent latex-coupled Spike protein with high-affinity and specificity, and a non-specific viral antibody reactive against coronavirus (CV) captures the fluorescent latex.

FIG. 4A is a Surface Plasmon Resonance (SPR) Sensorgram showing SARS-CoV-2 Spike protein binding affinity to human ACE2 receptor. FIG. 4B shows the structure of ACE2.

FIG. 5A is an exemplary schematic depiction of an ACE2-Fc fusion protein. FIG. 5B shows an exemplary amino acid sequence of the ACE2-Fc fusion protein.

FIG. 6 is a bar graph showing the signal (OD at 450 nm) from a double sandwich assay for nucleocapsid (N) protein when increasing salt (NaCl) concentrations were used in sample buffer. As shown, increasing salt concentrations in sample buffer increased recovery of nucleocapsid (N) protein from a HydraFlock® swab.

FIG. 7 is a bar graph showing the signal (OD at 450 nm) from a double sandwich assay for nucleocapsid (N) protein at increasing salt concentrations. Specifically, when 50 ng (left 4 bars) or 5 ng (right 4 bars) of N protein was assayed at the NaCl concentrations shown, signal intensity (OD at 450 nm) decreased with increasing salt concentration.

FIG. 8 is a bar graph showing the signal (OD at 450 nm) from a double sandwich assay for nucleocapsid (N) protein at varying N protein and salt concentrations over time. As shown, inclusion of salt in a sample buffer prevents the loss of signal over time. Looking at the right half of FIG. 8, when no salt is included in the buffer, at t=5, an almost 2-fold loss in signal is seen for the 100 ng/mL condition. The signal continues to decrease at the t==10, t=15 and t=20 time points. Looking at the left half of FIG. 8, although an overall lower signal was observed in conditions with 1M NaCl, there was no loss of signal from t==1 to t=20.

### DETAILED DESCRIPTION

The invention is based, in part, on the discovery that an ACE2 fusion protein is capable of functioning as a high-affinity binding moiety suitable for use in a detection assay, such as a sandwich assay, as described in further detail herein. It has been further discovered that the addition of salt (e.g., NaCl) to a sample buffer (1) increases recovery of viral protein from a sample and (2) and prevents prevent loss of viral protein on the surface of a microfluidic device, thereby improving the performance of a viral detection assay.

### I. Binding Moieties

(a) ACE2 Proteins

Angiotensin-converting enzyme-2 (ACE2) is a functional receptor for severe acute respiratory syndrome (SARS) coronaviruses and human coronavirus NL63. Surface Plasmon Resonance (SPR) Sensorgram data has shown that SARS-CoV-2 Spike S1 protein binding to human ACE2 receptor with kₐ of approximately 1.88×10⁵, similar to a kₐ observed with an antibody. (See, Kruse R. L. (2020), “Therapeutic strategies in an outbreak scenario to treat the novel coronavirus originating in Wuhan, China (version 2),” F1000Research, 9:72). The disclosure relates, in part, to the use of an ACE2 receptor or a variant or fragment thereof, as a binding moiety.

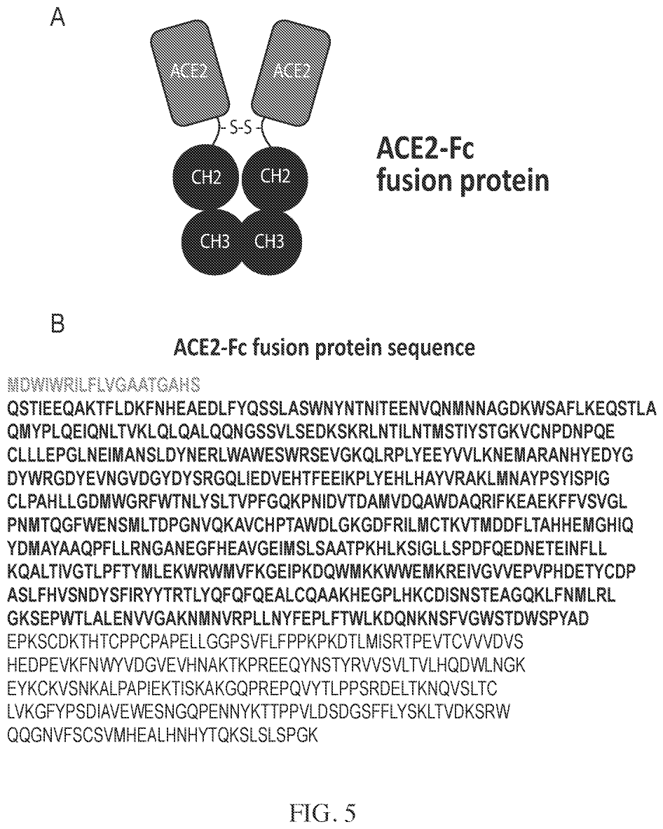

As shown in FIG. 5A-B, SEQ ID NO: 1 provides an amino acid sequence of an ACE2-Fc fusion protein comprising a secretion signal from a human IgG (amino acids 1-19, shown in bold and italics), a soluble ACE2 receptor portion (amino acids 20-617), and a human IgG1 hinge and Fc domain (amino acids 618-849, shown in bold).

SEQ ID NO: 1 is: [symbol]
QSTIEEQAKTFLDKFNHEAEDLFYQSSLA
SWNYNTNITEENVQNMNNAGDKWSAFLKEQSTLAQMYPLQEIQNLTVKL
QLQALQQNGSSVLSEDKSKRLNTILNTMSTIYSTGKVCNPDNPQECLLL
EPGLNEIMANSLDYNERLWAWESWRSEVGKQLRPLYEEYVVLKNEMARA
NHYEDYGDYWRGDYEVNGVDGYDYSRGQLIEDVEHTFEEIKPLYEHLHA
YVRAKLMNAYPSYISPIGCLPAHLLGDMWGRFWTNLYSLTVPFGQKPNI
DVTDAMVDQAWDAQRIFKEAEKFFVSVGLPNMTQGFWENSMLTDPGNVQ
KAVCHPTAWDLGKGDFRILMCTKVTMDDFLTAHHEMGHIQYDMAYAAQP
FLLRNGANEGFHEAVGEIMSLSAATPKHLKSIGLLSPDFQEDNETEINF
LLKQALTIVGTLPFTYMLEKWRWMVFKGEIPKDQWMKKWWEMKREIVGV
VEPVPHDETYCDPASLFHVSNDYSFIRYYTRTLYQFQFQEALCQAAKHE
GPLHKCDISNSTEAGQKLFNMLRLGKSEPWTLALENVVGAKNMNVRPLL
NYFEPLFTWLKDQNKNSFVGWSTDWSPYADEPKSCDKTHTCPPCPAPEL
LGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHE
ALHNHYTQKSLSLSPGK

In certain embodiments, the soluble ACE2 receptor comprises amino acids 20-617 of SEQ ID NO:1 or a variant or fragment thereof. In certain embodiments, the soluble ACE2 receptor or a variant or fragment thereof comprises an amino acid sequence having 80%, 85%, 86%, 87%, 88%, 89%, 90%, 91%, 92%, 93%, 94%, 95%, 96%, 97%, 98%, or 99% sequence identity to amino acids 20-617 of SEQ ID NO:1.

In certain embodiments, the soluble ACE2 receptor comprises amino acids 20-849 of SEQ ID NO:1 or a variant or fragment thereof. In certain embodiments, the soluble ACE2 receptor or a variant or fragment thereof comprises an amino acid sequence having 80%, 85%, 86%, 87%, 88%, 89%, 90%, 91%, 92%, 93%, 94%, 95%, 96%, 97%, 98%, or 99% sequence identity to amino acids 20-849 of SEQ ID NO:1.

In certain embodiments, the soluble ACE2 receptor comprises SEQ ID NO:1 or a variant or fragment thereof. In certain embodiments, the soluble ACE2 receptor or a variant or fragment thereof comprises an amino acid sequence having 80%, 85%, 86%, 87%, 88%, 89%, 90%, 91%, 92%, 93%, 94%, 95%, 96%, 97%, 98%, or 99% sequence identity to SEQ ID NO:1.

As used herein, the term “variant” of a soluble ACE2 receptor refers to a variant of a soluble ACE2 receptor that retains, for example, at least 10%, at least 20%, at least 30%, at least 40%, at least 50%, at least 60%, at least 70%, at least 80%, at least 90%, at least 95%, or 100% of the binding activity of the corresponding full-length, naturally occurring a soluble ACE2 receptor. Binding activity may be assayed by any method known in the art, including, for example, using an ELISA assay or a Biocore assay to measure binding of the soluble ACE2 receptor variant to an anti-ACE2 antibody or a natural ligand (e.g., angiotensin II). In certain embodiments, the variant comprises at least 50, at least 75, at least 100, at least 125, at least 150, at least 200, at least 250, at least 300, at least 350, at least 400, at least 450, or at least 500 consecutive amino acids present in a full-length, naturally occurring soluble ACE2 receptor.

In certain embodiments, the variant of a soluble ACE2 receptor comprises a conservative substitution relative to a soluble ACE2 receptor sequence disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution: p matrix (e.g., the PAM 250 matrix). In certain embodiments, the soluble ACE2 receptor comprises 1 or fewer, 2 or fewer, 3 or fewer, 4 or fewer, 5 or fewer, 6 or fewer, 7 or fewer, 8 or fewer, 9 or fewer, or 10 or fewer conservative substitutions.

As used herein, the term “fragment” of a soluble ACE2 receptor refers to a fragment of a soluble ACE2 receptor thereof that retains, for example, at least 10%, at least 20%, at least 30%, at least 40%, at least 50%, at least 60%, at least 70%, at least 80%, at least 90%, at least 95%, or 100% binding activity of the corresponding full-length, naturally occurring soluble ACE2 receptor. Binding activity may be assayed by any method known in the art, including, for example, using an ELISA assay or a Biocore assay to measure binding of the soluble ACE2 receptor variant to an anti-ACE2 antibody or a natural ligand (e.g., angiotensin II). In certain embodiments, the fragment comprises at least 50, at least 75, at least 100, at least 125, at least 150, at least 200, at least 250, at least 300, at least 350, at least 400, at least 450, or at least 500 consecutive amino acids present in a full-length, naturally occurring soluble ACE2 receptor.

Sequence identity may be determined in various ways that are within the skill of a person skilled in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36:290-300; Altschul et al., (1997) NUCLEIC ACIDS RES. 25:3389-3402, incorporated by reference herein) are tailored for sequence similarity searching. For a discussion of basic issues in searching sequence databases see Altschul et al., (1994) NATURE GENETICS 6:119-129, which is fully incorporated by reference herein. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference herein). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent blastp parameter settings may be Q==9; R==2; wink==1; and gapw==32. Searches may also be conducted using the NCBI (National Center for Biotechnology Information) BLAST Advanced Option parameter (e.g.: −G, Cost to open gap [integer]: default==5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; —W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty==10 and Gap Extension Penalty==0.1). A Bestfit comparison between sequences, available in the GCG package version 10.0, uses DNA parameters GAP=50 (gap creation penalty) and LEN=3 (gap extension penalty). The equivalent settings in Bestfit protein comparisons are GAP=8 and LEN=2.

In certain embodiments, the soluble ACE2 receptor or variant or fragment thereof is conjugated to an immunoglobulin Fc domain to form a fusion protein. As used herein, unless otherwise indicated, the term “immunoglobulin Fc domain” or “Fc domain” or “Fc” refers to a fragment of an immunoglobulin heavy chain constant region. An immunoglobulin Fc domain may include, e.g., immunoglobulin CH2 and CH3 domains. An immunoglobulin Fc domain may include, e.g., immunoglobulin CH2 and CH3 domains and an immunoglobulin hinge region. Boundaries between immunoglobulin hinge regions, CH2, and CH3 domains are well known in the art, and can be found, e.g., in the PROSITE database (available on the world wide web at prosite.expasy.org).

In certain embodiments, the immunoglobulin Fc domain is derived from a human IgG1, IgG2, IgG3, IgG4, IgA1, IgA2, IgD, IgE, and IgM Fc domain. A single amino acid substitution (S228P according to Kabat numbering; designated IgG4Pro) may be introduced to abolish the heterogeneity observed in recombinant IgG4 antibody. See Angal, S. et al. (1993) MOL. IMMUNOL. 30:105-108.

In certain embodiments, the immunoglobulin Fc domain is derived from a human IgG1 isotype or another isotype that elicits antibody-dependent cell-mediated cytotoxicity (ADCC) and/or complement mediated cytotoxicity (CDC). In certain embodiments, the immunoglobulin Fc domain is derived from a human IgG1 isotype (e.g., amino acids 618-849 of SEQ ID NO: 1).

In certain embodiments, the immunoglobulin Fc domain is derived from a human IgG4 isotype or another isotype that elicits little or no antibody-dependent cell-mediated cytotoxicity (ADCC) and/or complement mediated cytotoxicity (CDC). In certain embodiments, the immunoglobulin Fc domain is derived from a human IgG4 isotype.

In certain embodiments, the immunoglobulin Fc domain comprises either a “knob” mutation, e.g., T366Y, or a “hole” mutation, e.g., Y407T, for heterodimerization with a second polypeptide (residue numbers according to EU numbering, Kabat, E. A., et al. (1991) SEQUENCES OF PROTEINS OF IMMUNOLOGICAL INTEREST, FIFTH EDITION, U.S. Department of Health and Human Services, NIH Publication No. 91-3242). For example, in certain embodiments, the immunoglobulin Fc domain is derived from a human IgG1 Fc domain and comprises a Y407T mutation. In certain embodiments, the immunoglobulin Fc domain is derived from a human IgG1 Fc domain and comprises a T366Y mutation.

In certain embodiments, the immunoglobulin Fc domain is modified to prevent to glycosylation of the Fc domain. For example, in certain embodiments, the immunoglobulin Fc domain is derived from a human IgG1 Fc domain and comprises a mutation at position N297, for example, an N297A mutation (residue numbers according to EU numbering, Kabat, E. A., et al., supra).

(b) Antibodies

In certain embodiments, a binding moiety comprises an antibody that binds a viral protein as described herein. As used herein, unless otherwise indicated, the term “an anti-coronavirus antibody” is understood to mean an antibody that binds to a viral protein of a coronavirus. As used herein, unless otherwise indicated, the term “antibody” is understood to mean an intact antibody (e.g., an intact monoclonal antibody), or an antigen-binding fragment thereof, such as a Fc fragment of an antibody (e.g., an Fc fragment of a monoclonal antibody), or an antigen-binding fragment of an antibody (e.g., an antigen-binding fragment of a monoclonal antibody), including an intact antibody, antigen-binding fragment, or Fc fragment that has been modified, engineered, or chemically conjugated. Examples of antigen-binding fragments include Fab, Fab′, (Fab′)2, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). In certain embodiments, the antigen-binding fragment retains, for example, at least 10%, at least 20%, at least 30%, at least 40%, at least 50%, at least 60%, at least 70%, at least 80%, at least 90%, at least 95%, or 100% binding activity of the corresponding full-length antibody.

In certain embodiments, monoclonal antibodies are used as capture and/or detection moieties. A monoclonal antibody refers to an antibody that is derived from a single clone, including any eukaryotic, prokaryotic, or phage clone. The monoclonal antibody may comprise, or consist of, two proteins, i.e., heavy and light chains. The monoclonal antibody can be prepared using one of a wide variety of techniques known in the art including the use of hybridoma, recombinant, and phage display technologies, or a combination thereof.

Monoclonal antibodies capable of binding a viral protein may be prepared using any known methodology, including the seminal hybridoma methods, such as those described by Kohler and Milstein (1975), Nature. 256:495. In a hybridoma method, a mouse, hamster, or other appropriate host animal is immunized with an immunizing agent to elicit lymphocytes that produce or are capable of producing antibodies that will specifically bind to the immunizing agent. Alternatively, the lymphocytes may be immunized in vitro.

The immunizing agent will typically include at least a portion of the viral protein. For example, synthetic polypeptide or recombinant polypeptide comprising a portion of a spike protein (S), nucleocapsid protein (N), envelope protein (E), membrane protein (M), or Hemagglutinin-esterase dimer protein (HE) may be used. The immunizing agent may be administered to a mammal with or without adjuvant according to any of a variety of standard methods.

In certain embodiments, either the capture moiety or the detection moiety comprises the SARS-CoV/SARS-CoV-2 Nucleocapsid Antibody, Mouse Mab from Sino Biological, Inc. (40143-MM05), or a variant or fragment thereof. In certain embodiments, either the capture moiety or the detection moiety comprises the SARS-CoV/SARS-CoV-2 Nucleocapsid Antibody, Rabbit Fab from LumiraDx UK Ltd. (SD-QMS-WI-30066).

### II. Methods of Making a Polypeptide

Methods for producing polypeptides, for example, a soluble ACE2 receptor or variant or fragment thereof, are known in the art. In certain embodiments, the polypeptides are chemically synthesized using techniques such as liquid-phase or solid-phase peptide synthesis.

In other embodiments, DNA molecules encoding the polypeptide can be synthesized chemically or by recombinant DNA methodologies. For example, the DNA sequence encoding the polypeptide can be cloned using polymerase chain reaction (PCR) techniques, using the appropriate synthetic nucleic acid primers. The resulting DNA molecules can be ligated to other appropriate nucleotide sequences, including, for example, expression control sequences, to produce conventional gene expression constructs (i.e., expression vectors) encoding the desired polypeptide. Production of defined gene constructs is within routine skill in the art.

Nucleic acids encoding desired polypeptides can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells. Transformed host cells can be grown under conditions that permit the host cells to express the genes that encode the polypeptides.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. The gene construct can be introduced into eukaryotic host cells using conventional techniques.

A polypeptide can be produced by growing (culturing) a host cell transfected with an expression vector encoding the polypeptide, under conditions that permit its expression.

Following expression, the polypeptide can be harvested and purified or isolated using techniques known in the art, e.g., affinity tags such as glutathione-S-transferase (GST) or histidine tags.

### III. Detection of Viral Protein

According to the methods of the invention, the viral protein may be detected and/or quantitated in a sample using a first and second binding moiety (e.g., a soluble ACE receptor, or a variant or fragment thereof, and an anti-coronavirus antibody or fragment thereof). Viral proteins suitable for detection according to the methods disclosed herein include any protein from a virus, wherein the virus or a portion thereof (e.g., a viral protein) is capable of binding to a soluble ACE2 receptor (e.g., a human ACE2 receptor). Exemplary viruses include HCoV-NL63, SARS-CoV, and SARS-CoV-2 (see, e.g., the NCBi entry for ACE2 angiotensin converting enzyme 2 (Homo sapiens (human)) at nih.gov). Exemplary viral proteins include spike protein (S), nucleocapsid protein (N), envelope protein (E), membrane protein (M), and Hemagglutinin-esterase dimer protein (HE).

Spike protein (S) is heavily glycosylated, utilizes an N-terminal signal sequence to gain access to the ER and mediate attachment to host receptors. It is the largest structure and makes the distinct spikes on the surface of the virus. For most coronaviruses, S protein is cleaved by a host cell furin-like protease into two separate polypeptides S1 and S2.

Nucleocapsid protein (N) binds to RNA in vitro and is heavily phosphorylated. N proteins binds the viral genome in a beads on a string type conformation. This protein may help tether the viral genome to replicase-transcriptase complex (RTC), and subsequently package the encapsulated genome into viral particles.

Envelope protein (E) is found in small quantities in within the virus. It is most likely a transmembrane protein and with ion channel activity. The protein facilitates assembly and release of the virus and has other functions such as ion channel activity. It is not necessary for viral replication but it is for pathogenesis.

Membrane protein (M) is the most abundant structural protein. It does not contain signal sequence and exists as a dimer in the virion. It may have two different conformations to enable it to promote membrane curvature as well as bind to nucleocapsid.

Hemagglutinin-esterase dimer protein (HE) is present in a subset of betacoronaviruses. The protein binds sialic acids on surface glycoproteins. The protein activities are thought to enhance S protein-mediated cell entry and virus spread through the mucosa.

The methods and compositions of the present invention can be used to detect the concentration of viral protein in a sample. The sample may be from, e.g., blood-based sample such as blood, plasma, or serum, e.g., wherein the sample comprises or consists essentially of serum and/or plasma. The sample may be from, e.g., a nasal or nasopharyngeal swab specimen or saliva sample and/or contained in Universal Transport Media (UTM) or Viral Transport Media (VTM). Methods of obtaining a body fluid or tissue sample from a subject are known to those skilled in the art.

In embodiments, a sample is or has been exposed to a lysing reagent that comprises a sufficient amount of an exonuclease to release a viral protein (e.g., nucleocapsid protein) from RNA of the virus. Releasing the protein from the RNA increases the amount of protein available to participate in a reaction (e.g., an immunological reaction) to determine the presence of the protein in a sample. In certain embodiments, the exonuclease is present in an amount of about 0.002 to about 1 U/mL. An exemplary exonuclease is Benzonase® nuclease.

As described in Example 4 herein, the addition of salt (e.g., NaCl) to a sample (e.g., in a sample buffer) increases recovery of viral protein from a sample and prevents prevent loss of viral protein on the surface of a microfluidic device, thereby improving the performance of a viral detection assay.

Accordingly, in certain embodiments of the methods disclosed herein, a sample is contacted with a buffer comprising a salt solution prior to subjecting the sample to the double binding moiety sandwich assay. In certain embodiments, the salt is present at a concentration of at least about 0.2 M, at least about 0.3 M, or at least about 0.4 M. The salt concentration may be about 1.2 M or less, about 1.1 M or less, about 1.0 M or less, or about 0.9 M or less. In certain embodiments, the salt concentration is between about 0.2 M and about 0.9 M, about 0.2 M and about 1.0 M, between about 0.2 M and about 1.1 M, between about 0.2 M and about 1.2 M, between about 0.3 M and about 0.9 M, about 0.3 M and about 1.0 M, between about 0.3 M and about 1.1 M, between about 0.3 M and about 1.2 M, between about 0.4 M and about 0.9 M, about 0.4 M and about 1.0 M, between about 0.4 M and about 1.1 M, or between about 0.4 M and about 1.2 M.

In certain embodiments, the sample is a liquid sample comprising a buffer, wherein the liquid sample includes salt at a concentration of at least about 0.2 M, at least about 0.3 M, or at least about 0.4 M. The salt concentration may be about 1.2 M or less, about 1.1 M or less, about 1.0 M or less, or about 0.9 M or less. In certain embodiments, the salt concentration is between about 0.2 M and about 0.9 M, about 0.2 M and about 1.0 M, between about 0.2 M and about 1.1 M, between about 0.2 M and about 1.2 M, between about 0.3 M and about 0.9 M, about 0.3 M and about 1.0 M, between about 0.3 M and about 1.1 M, between about 0.3 M and about 1.2 M, between about 0.4 M and about 0.9 M, about 0.4 M and about 1.0 M, between about 0.4 M and about 1.1 M, or between about 0.4 M and about 1.2 M.

Exemplary salts include chloride salts such as sodium or potassium chloride and combinations thereof.

According to the methods of the invention, viral protein is detected and/or quantified using a “sandwich” assay, such as ELISA. In this embodiment, a first and second binding moiety (e.g., a soluble ACE receptor, or a variant or fragment thereof, and an anti-coronavirus antibody or fragment thereof) that specifically bind to non-overlapping sites (“epitopes”) on a viral protein are used. Typically, a “capture moiety” is a binding moiety immobilized on a solid surface (e.g., a bead, particle, or channel of a microfluidic device) where it binds with and captures the viral protein. A second binding moiety is detectably labeled, for example, with a fluorophore, enzyme, or colored particle (“detection moiety”), such that binding of the detection moiety to the viral protein-capture moiety-complex indicates that the viral protein has been captured. The intensity of the signal is proportional to the concentration of viral protein in the sample.

Such assay procedures can be referred to as two-site immunometric assay methods, i.e., “sandwich immunoassays.” As is known in the art, the capture and detection moieties can be contacted with the test sample simultaneously or sequentially. Sequential methods, sometimes referred to as the “forward” method, can be accomplished by incubating the capture moiety with the sample, and adding the labeled detection moiety at a predetermined time thereafter. Alternatively, the labeled detection moiety can be incubated with the sample first and then the sample can be exposed to the capture moiety (sometimes referred to as the “reverse” method). After any necessary incubation(s), which may be of short duration, the label is detected and may also be measured. Such assays may be implemented in many specific formats known to those of skill in the art, including through use of various high throughput clinical laboratory analyzers or with point of care or home testing devices.

In certain embodiments, viral protein is detected and/or quantified within a microfluidic device, such as that described in International Patent Application No. PCT/US2021/013325. In certain embodiments, the microfluidic device (e.g., microfluidic strip) is configured to perform an assay to detect an viral protein, e.g., a SARS-CoV-2 protein, in a sample. The microfluidic strip can include a microfluidic channel network including a sample application port and an analysis channel in fluidic communication therewith. The analysis channel includes a first binding moiety and a second binding moiety that bind to a viral protein, such as a SARS CoV-2 viral protein.

In certain embodiments, reagents for detecting a virus (e.g., a viral protein such as a coronavirus protein) can be present in one microchannel and control reagents can be present in another microchannel of the same device.

In some embodiments, one of the first binding moiety and the second binding moiety is bound to, or is configured to bind to a capture agent (e.g., a surface, such as a surface of a channel of the microchannel network, or a particle, such as a magnetic particle) and the other of the first binding moiety and the second binding moiety is bound to or is configured to bind to a detectable label. For example, a capture moiety may comprise a conjugate of (i) a soluble ACE2 receptor or variant or fragment thereof and (ii) a binding agent configured to bind to a surface or particle such as a magnetic particle. For example, the conjugate may include one of biotin, and avidin or streptavidin and the particle or surface may include the other of biotin and avidin or streptavidin, e.g., the capture moiety may be a conjugate of (i) a soluble ACE2 receptor or variant or fragment thereof, and (ii) biotin, and the microfluidic strip may further include a particle, e.g., a magnetic particle, conjugated to streptavidin. The second reagent may be a conjugate including (i) an antibody to a viral protein (e.g., a SARS-CoV-2 viral protein) and (ii) a detectable label such as a fluorescent particle, e.g., a fluorescent latex particle. In certain embodiments, the soluble ACE2 receptor or variant or fragment thereof binds to a different epitope on the SARS-CoV-2 antigen than does the SARS-CoV-2 antibody.

In certain embodiments, the capture moiety comprises a conjugate including (i) an antibody to a viral protein (e.g., a SARS-CoV-2 viral protein) and (ii) a binding agent configured to bind to a surface or particle such as a magnetic particle (e.g., via biotin and avidin or streptavidin) and/or the detection moiety comprises a conjugate including (i) a soluble ACE2 receptor or variant or fragment thereof and (ii) a detectable label such as a fluorescent particle, e.g., a fluorescent latex particle.

In embodiments, a method of performing an assay to detect an antigen, e.g., a SARS-CoV-2 antigen includes combining a liquid sample, e.g., a nasal, nasopharyngeal, or saliva-based sample, which may be present in Universal Transport Media (UTM) or Viral Transport Media (VTM), suspected of containing such an antigen with a first reagent including a soluble ACE2 receptor or variant or fragment thereof and a second reagent including a second antibody to a viral (e.g., nucleocapsid) and determining the presence and/or amount of a complex including the first binding moiety, the viral protein, and the second binding moiety.

In some embodiments, the first and second binding moieties are disposed within an analysis channel of the microfluidic channel network. A distal portion of the analysis channel may include a gas bladder and the method may include compressing, decompressing and/or oscillating the gas bladder as disclosed herein to manipulate the liquid sample e.g., to move the liquid sample and/or mix the liquid sample and reagents as disclosed herein. The method may include magnetically retaining complexes the first binding moiety, the viral protein, and the second binding moiety in a detection zone of the microfluidic channel network prior to detecting the complexes. The method may include expelling sample liquid from the detection zone as disclosed herein prior to the detecting step.

In another embodiment, a lateral flow device may be used in the sandwich format, wherein the presence of viral protein above a baseline sensitivity level in a biological sample will permit formation of a sandwich interaction upstream of or at the capture zone in the lateral flow assay. See, for example, U.S. Pat. No. 6,485,982. The capture zone as used herein may contain capture moieties, suitable for capturing viral protein, or immobilized avidin or the like for capture of a biotinylated complex. See, for example, U.S. Pat. No. 6,319,676. The device may also incorporate a luminescent label suitable for capture in the capture zone, the concentration of viral protein being proportional to the intensity of the signal at the capture site. Suitable labels include fluorescent labels immobilized on polystyrene microspheres. Colored particles also may be used.

Other assay formats that may be used in the methods of the invention include, but are not limited to, flow-through devices. See, for example, U.S. Pat. No. 4,632,901. In a flow-through assay, an binding moiety (antibody) is immobilized to a defined area on a membrane surface. This membrane is then overlaid on an absorbent layer that acts as a reservoir to pump sample volume through the device. Following immobilization, the remaining protein-binding sites on the membrane are blocked to minimize non-specific interactions. In operation, a biological sample is added to the membrane and filters through, allowing any analyte specific to the binding moiety in the sample to bind to the immobilized antibody. In a second step, a labeled second binding moiety may be added or released that reacts with captured marker to complete the sandwich. Alternatively, the second binding moiety can be mixed with the sample and added in a single step. If viral protein is present, a colored spot develops on the surface of the membrane.

The most common enzyme immunoassay is the “Enzyme-Linked Immunosorbent Assay (ELISA).” ELISA is a technique for detecting and measuring the concentration of an antigen using a labeled (e.g., enzyme linked) form of the antibody. There are different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. (1984), J. CLIN. CHEM. CLIN. BIOCHEM. 22:895-904.

In a “sandwich ELISA,” an first binding moiety is linked to a solid phase (i.e., a microtiter plate) and exposed to a biological sample containing antigen (e.g., viral protein). The solid phase is then washed to remove unbound antigen. A labeled antibody (e.g., enzyme linked) is then bound to the bound antigen, forming an antibody-antigen-antibody sandwich. Examples of enzymes that can be linked to the antibody are alkaline phosphatase, horseradish peroxidase, luciferase, urease, and β-galactosidase. The enzyme-linked antibody reacts with a substrate to generate a colored reaction product that can be measured. This measurement can be used to derive the concentration of viral protein present in a sample, for example, by comparing the measurement to a viral protein standard curve. In certain embodiments, a fluorescent molecule or other marker is used to label the antibody instead of an enzyme.

### IV. Capture Agents

The important property of the capture agent is that it provides a means of separation from the remainder of the test mixture. Accordingly, as is understood in the art, the capture agent can be introduced to the assay in an already immobilized or insoluble form, that is, a form which enables separation of the complex from the remainder of the test solution. Alternatively, immobilization may be done by capture of an immune complex comprising a viral protein subsequent to introduction of a soluble form of the capture agent to the sample. Examples of immobilized capture agent are antibodies covalently or noncovalently attached to a solid phase such as a magnetic particle, a latex particle, a microtiter plate well, a membrane, a chip, a bead, a cuvette, an array, or other reaction vessel or holder. Examples of a soluble capture agent is an antibody which has been chemically modified with a ligand, e.g., a hapten, biotin, or the like, and which acts as a hook to permit selective capture of complex including a viral protein. Methods of coupling the capture agent to a solid phase are well known in the art. These methods can employ bifunctional linking agents, for example, or the solid phase can be derivatized with a reactive group, such as an epoxide or an imidizole, that will bind the molecule on contact. Bispecific capture reagents against different target proteins can be mixed in the same place, or they can be attached to solid phases in different physical or addressable locations.

### V. Labels

According to the methods of the invention, the label used can be selected from any of those known conventionally in the art. Preferred labels are those that permit more precise quantitation. Examples of labels include but are not limited to a fluorescentmoiety, an enzyme, an electrochemically active species, a radioactive isotope, a chemiluminescent molecule, a latex or gold particle, a detectable ligand (e.g., detectable by secondary binding of a labeled binding partner for the ligand), etc. In a preferred embodiment, the label is an enzyme or a fluorescent molecule. Methods for affixing the label to the antibody are well known in the art, and include covalent and non-covalent linkage.

In one embodiment, a detection antibody can be labeled with a fluorescent compound. When the fluorescently labeled detection antibody is exposed to light of the proper wavelength, its presence can then be detected by the fluorescence emitted. Among the most commonly used fluorescent labeling compounds are Cy3 and Cy5 (water-soluble fluorescent dyes of the cyanine dye family—“Cy” dyes), fluorescein isothiocyanate, rhodamine, phycoerytherin, phycocyanin, allophycocyanin, o-phthalaldehyde and fluorescamine. In certain embodiments, the fluorescence compound is green fluorescent protein, red fluorescent protein, or a variant thereof.

In another embodiment, the detection moiety is detectably labeled by linking the antibody to an enzyme. The enzyme, in turn, when exposed to its substrate, will react with the substrate in such a manner as to produce a chemical moiety which can be detected, for example, by spectrophotometric, fluorometric or visual means. Enzymes which can be used to detectably label the detection antibody of the present invention include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In certain embodiments, the detection antibody is conjugated to alkaline phosphatase, and binding of the detection antibody to the polypeptide-capture antibody complex is detected by adding, for example, disodium 2-chloro-5-(4-methoxyspiro[1,2-dioxetane-3,2′-(5-chlorotricyclo[3.3.1.13.7]decan])-4-yl]-1-phenyl phosphate and measuring chemiluminescence.

Detection may also be accomplished using a radioactively labeled antibody. It is then possible to detect the antibody through the use of radioimmune assays. The radioactive isotope can be detected by such means as the use of a gamma counter or a scintillation counter or by autoradiography. Isotopes which are particularly useful for the purpose of the present invention are 3H, 131I, 35S, 14C, and preferably 125I.

A binding moiety also can be detectably labeled by coupling it to a chemiluminescent compound. The presence of the chemiluminescent compound-antibody complex is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The invention will be more readily understood by reference to the following examples, which are included merely for purposes of illustration of certain aspects and embodiments of the present invention, and is not intended to limit the invention. The terms “bead” and “particle” are used synonymously herein.

### Example 1—Assay to Measure the Subpopulation of SARS-CoV-2 Specific IgM

### Assay 1 Outline

In this example, magnetic beads coupled to anti-human-IgMs capture a population of IgMs present in a sample, e.g., a swab, e.g., serum. In an individual positive for coronavirus SARS-CoV-2, i.e., an individual with COVID-19, a subpopulation of said IgMs is directed against SARS-CoV-2 protein. Latex particles modified with recombinant Spike protein, a protein on the surface of the virus, binds to the subpopulation of anti-SARS-CoV-2 antibodies bound to magnetic beads. A magnetic pull-down is performed, followed by washing unbound latex, in order to capture only magnetic bead-bound latex, i.e. those bound to Spike protein.

This immunoassay measures immune response to SARS-CoV-2 infection that comes either in the form of IgMs (early immune response) or IgG (later, more mature immune response). Higher immune response against SARS-CoV-2 gives higher signal.

### Assay 1 Description

The assay is conducted using magnetic beads bearing surface anti-human IgG and/or IgM antibodies and Spike-protein modified fluorescent latex.

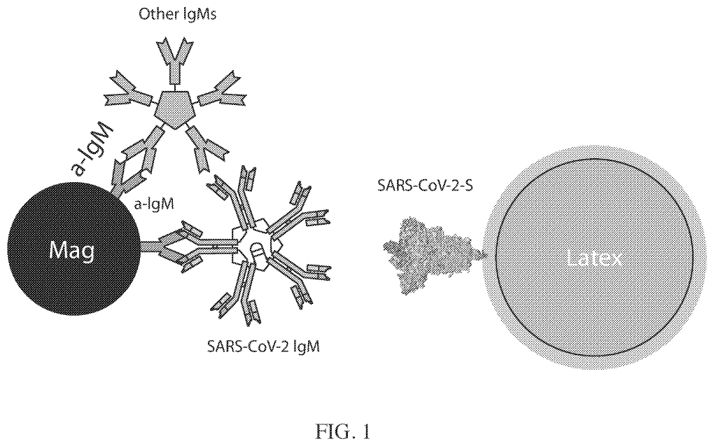

To perform the assay, a sample, e.g., an infected sample, is introduced into a first assay device chamber, thereby reconstituting dry anti-human IgG and/or IgM magnetic beads within the chamber. Following reconstitution, the sample is incubated with the beads for 60 seconds, followed by mixing in the same chamber for 90 seconds. The magnetic beads capture the entire population of IgGs and/or IgMs from the infected serum; a certain subpopulation of the captured Abs contains anti-Spike protein Abs. The magnetic beads are then moved to a second chamber containing dry labeled Spike-protein modified fluorescent latex. The Spike-protein reagent is made of dyed polystyrene particles approximately 1 micron in diameter, coated with Spike protein. The resulting suspension is incubated for 60 seconds and is then mixed for 90 seconds. Spike-protein labeled fluorescent latex is bound by the magnetic beads carrying anti-Spike antibodies. The magnetic beads are immobilized with a magnetic field for 120 seconds, followed by a 3 minute wash. Fluorescent intensity is determined. A schematic summary of the assay is shown in FIG. 1.

The resulting signal from the immobilized beads proportional to the specific immune response against coronavirus SARS-CoV-2. A higher fluorescent signal is indicative of a higher immune response.

### Example 2—Assay to Measure the Direct Immune Response to Spike Protein

### Assay 2 Outline

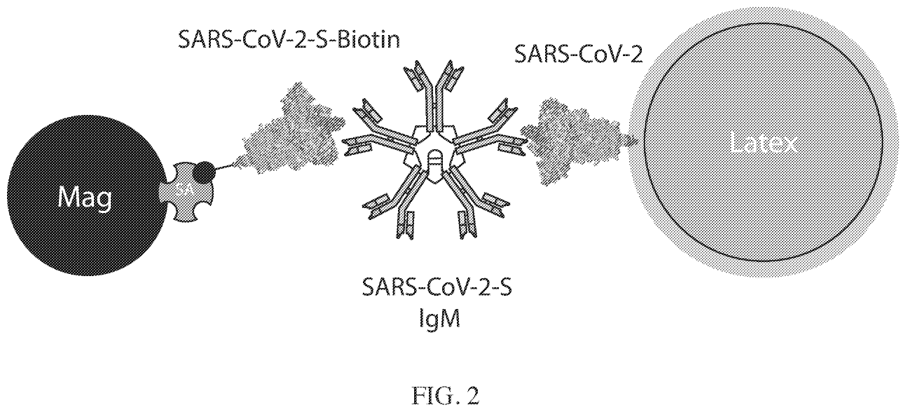

In this example, as depicted in the schematic in FIG. 2, one SARS-CoV-2 Spike protein is biotinylated and another SARS-CoV-2 Spike protein is conjugated to fluorescent latex. In serum in which SARS-CoV-2 is present, the two Spike proteins form a bridged complex with anti-Spike antibodies. The resultant immunocomplex is then captured by streptavidin-coated magnetic beads.

A higher immune response against coronavirus SARS-CoV-2 yields a higher fluorescent signal in the assay.

### Assay 2 Description

To conduct the assay, a sample, e.g., a liquid sample is introduced into a first assay device chamber. Exemplary liquid samples include a nasopharyngeal swab, a throat swab, or combination thereof or an eluant, e.g., in buffer, of such a swab. Other examples include blood-based samples such as whole blood, serum, or plasma. The first assay device chamber contains dry, e.g., lyophilized, biotinylated Spike protein admixed with Spike protein modified fluorescent beads. The resulting mixture is incubated for 60 seconds. After incubation, the sample and beads are mixed in the same chamber for 90 seconds. The fluorescent Spike-protein reagent is made of dyed polystyrene particles ranging from about 0.5 micron to about 1 micron in diameter and is coated with Spike protein. Any Spike-protein specific antibody known in the art is expected to form a bridge between the biotinylated and fluorescent reagents due to the multivalent nature of an antibody.

The biotinylated-fluorescent complexes are subsequently moved to a second chamber containing dry magnetic beads coupled to streptavidin. The resulting solution is incubated for 60 seconds, then mixed for 90 seconds. The streptavidin coated magnetic beads capture biotinylated fluorescent bridge complexes. The magnetic beads are immobilized with a magnetic field for 120 seconds, followed by a 3 minute was such that any unbound fluorescence is washed away. Fluorescent intensity is determined.

The resulting signal from the immobilized beads is proportional to the total immune response against coronavirus SARS-CoV-2. The assay is expected to measure total immune response against SARS-CoV-2 (both IgG and IgM), providing highly sensitive, highly specific detection of infectivity.

### Example 3—Assay to Measure the Viral Titer of SARS-CoV-2

### Assay 3 Outline

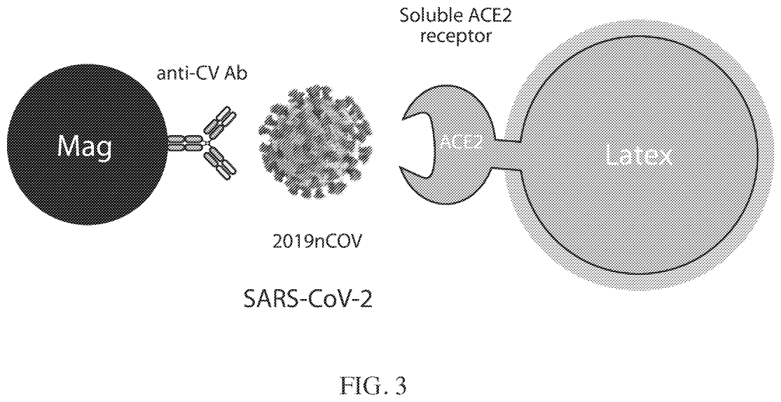

In this example, an embodiment of which is shown in the schematic depicted in FIG. 3, magnetic beads modified with anti-coronavirus antibody capture the whole virus (or viral debris, not shown). The antibody is a non-specific viral antibody that displays cross-reactivity with coronavirus SARS-CoV-2 (“anti-CV Ab”). For example, the viral antibody can be to a spike protein (S), a nucleocapsid protein (N), an envelope protein (E), a membrane protein (M), or a hemagglutinin-esterase dimer protein (HE).

Fluorescent latex coupled to soluble angiotensin-converting enzyme 2 (ACE2) receptor captures SARS-CoV-2 virus by binding Spike proteins with high, antibody-like affinity.

This sandwich immunoassay measures Spike-specific virus titer. The assay is useful for determining Spike-specific virus titer in infected individuals. The assay may also be used to determine the absence of Spike-specific virus in uninfected individuals.

### Assay 3 Description

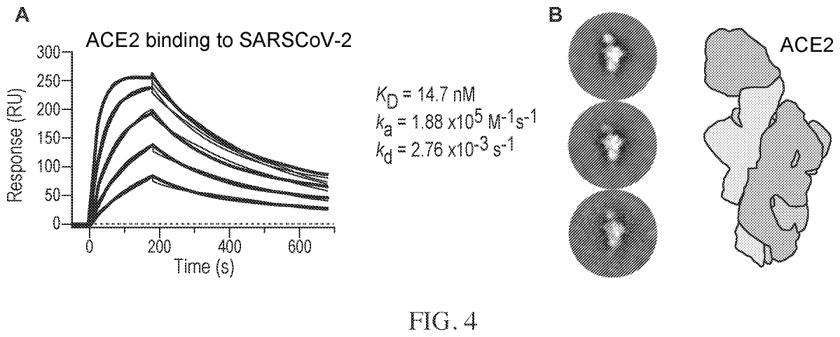

During infection, Spike protein on the surface of SARS-CoV-2 interacts with ACE2 receptor on the host cells, e.g., mammalian cells, e.g., human cells, for cellular entry. FIG. 4A shows an Surface Plasmon Resonance (SPR) Sensorgram showing SARS-CoV-2 Spike S1 protein binding to human ACE2 receptor with kₐ of approximately 1.88×10⁵, similar to a kₐ observed with an antibody. The structure of ACE2 is shown in FIG. 4B (see Kruse R. L. (2020), supra).

In this assay, the ACE2 Receptor is used as a binding agent of SARS-CoV-2 instead of an antibody. The ACE2 Receptor is modified with the addition of an Fc domain, as shown in FIG. 5A, using standard molecular biology techniques known in the art. The addition of the Fc domain facilitates dimerization of two ACE2 domains, stabilizes ACE2, and improves the k_{d} value. Additionally, the Fc domain also provides a conjugation site to facilitate the orientation of ACE2 on the labeled particles. An exemplary sequence of ACE2 modified with an Fc domain is shown in FIG. 5B (see Kruse R. L. (2020), supra).

The assay is conducted using magnetic beads bearing anti-viral protein antibodies and ACE2 receptor modified fluorescent latex. The fluorescent ACE2 reagent is comprised of dyed polystyrene particles ranging from about 0.5 micron to about 1 micron in diameter, coupled to soluble human ACE2 receptor. To conduct the assay, a sample (e.g., a liquid as described above) is introduced into a first assay device chamber, thereby reconstituting the dry ACE2 fluorescent beads within the chamber, and incubated for 60 seconds; following incubation, the resultant composition is mixed in the same chamber for 90 seconds. The ACE2 modified beads capture both whole virus or viral debris from the sample by binding to Spike surface protein.

The fluorescent bead complexes are subsequently moved to a second chamber containing dry, labeled magnetic beads modified with commercial anti-coronavirus antibodies, reconstituting the beads. The resultant suspension is incubated for 60 seconds, then mixed for 90 seconds. Fluorescent latex carrying viral debris is bound by the magnetic beads carrying anti-virus antibodies. The magnetic beads are then immobilized with a magnetic field for 120 seconds followed by a 3 minute wash such that any unbound fluorescent latex is washed away. Fluorescent intensity is determined. The assay may also be performed in reverse, whereby a sample, e.g., an infected sample, is bound with magnetic beads first, and subsequently bound to fluorescent latex.

The resulting fluorescent signal from the immobilized beads is proportional to the virus titer present in the sample and proportional to the total immune response against coronavirus SARS-CoV-2. The assay is expected to measure total immune response against SARS-CoV-2 (both IgG and IgM), providing highly sensitive, highly specific detection.

### Example 4—Addition of Salt to Sample Buffer Increases Recovery of Viral Protein

This example shows that the addition of salt (e.g., NaCl) to a sample buffer increases recovery of viral protein from a sample and prevents prevent loss of viral protein on the surface of a microfluidic device, thereby improving the performance of a viral detection assay.

100 ng/mL or 5 ng/mL SARS-CoV-2 nucleocapsid protein (N) was prepared in Tauns buffer (100 mM Tris, 1% Tween 20; TAUNS Laboratories Inc., Shizuoka, Japan) with varying NaCl concentrations. 50 μL blank Tauns buffer was dispensed on a clean dry HydraFlock® swab (Puritan, Guilford, Me.). The swab was dipped into an extraction tube with a flexible wall for squeezing the swab. The extraction tube contained 300 μL of 100 ng/mL or 5 ng/mL SARS-CoV-2 N protein-containing buffer. The swab was swirled for 15 seconds and wrung out by gently pressing it between the walls of the extraction tube. 100 μL of each sample condition was tested by double sandwich assay to detect SARS-CoV-2 N protein. The double sandwich assay used anti-N protein antibody-coated microtiter wells and a horseradish peroxidase-labeled N protein monoclonal antibody (Creative Diagnostics®, Shirley, N.Y.).

As shown in TABLE 1, recovery of N protein increased with increasing NaCl concentrations.

TABLE 1

OD at 450 nm

| | Covid-19 NP | Recovery after single |
|---|---|---|
| [NaCl] | 50 ng/mL | swab in 100 ng/mL |

| | | | |
|---|---|---|---|
| 0 | mM | 2.923 | 0.128 |
| 150 | mM | 1.023 | 0.22 |
| 500 | mM | 0.668 | 0.501 |
| 1000 | mM | 0.59 | 0.848 |

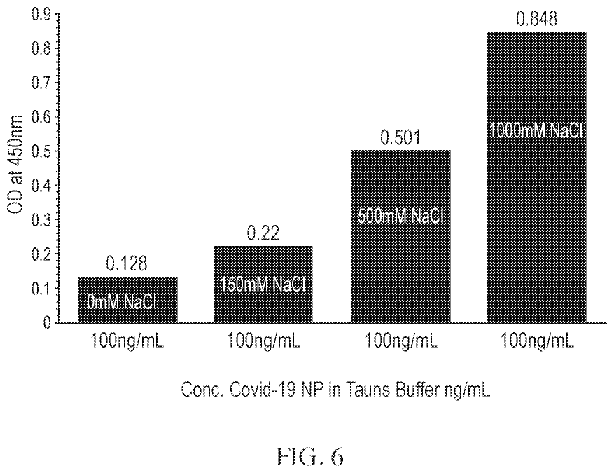

As shown in FIG. 6, increasing salt (NaCl) concentrations increased recovery of N protein.

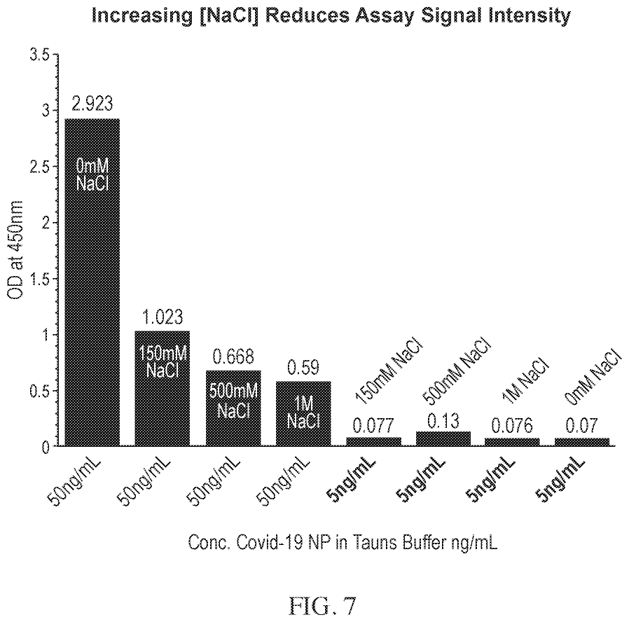

It was also discovered that high salt concentration reduced signal intensity in a double sandwich assay to detect SARS-CoV-2 N protein. As shown in in FIG. 7, when 50 ng (left 4 bars) or 5 ng (right 4 bars) of N protein was assayed at the NaCl concentrations shown, signal intensity (OD at 450 nm) decreased with increasing salt concentration.

It was also discovered that the addition of salt to sample buffer appeared to inhibit adhesion of viral protein to surfaces used in a microfluidic device, thereby increasing signal in a double sandwich assay.

Specifically, 100 ng/mL or 70 ng/mL SARS-CoV-2 nucleocapsid protein (N) was made in buffer with or without 1M NaCl. 200 μL of above buffer with protein was dispensed on the hydrophilic side of PET 9984 (3M, St. Paul, Minn.) and incubated at RT for t=1 and t=5. 100 μL of each condition was tested by double sandwich assay to detect SARS-CoV-2 N protein. The experiment was then repeated but incubated for longer time points, t=10, t=15, and t=20.

PET 9984 is a film coating that can be used in a microfluidic device. It has an anionic surfactant coating on its hydrophilic surface. Anionic surfactants have a negative charge on their hydrophilic end.

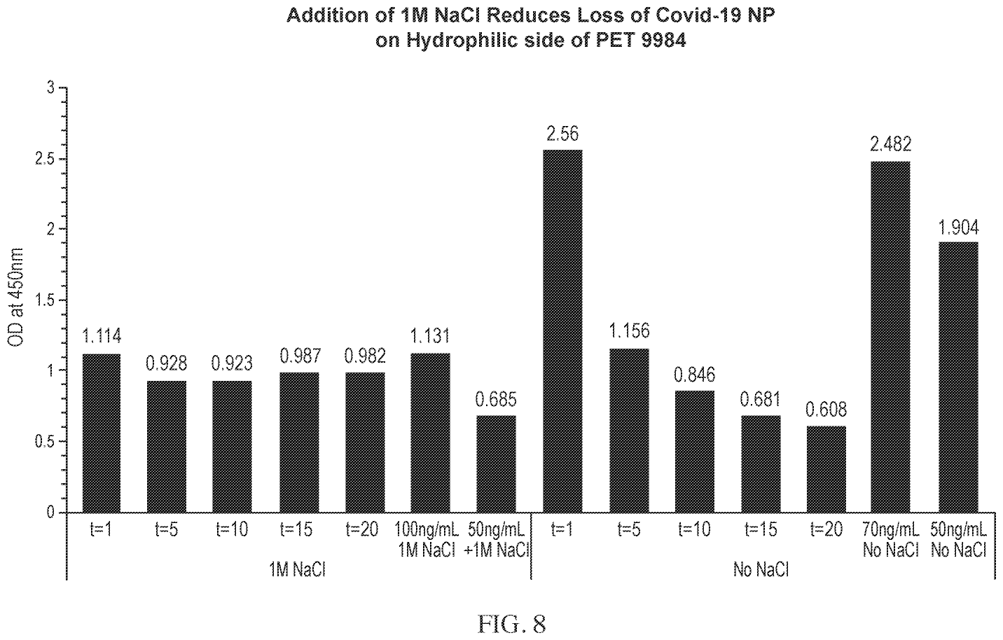

Looking at the right half of FIG. 8, when no salt is included in the buffer, at t==5, an almost 2-fold loss in signal is seen for the 100 ng/mL condition. The signal continues to decrease at the t=10, t=15 and t=20 time points. Looking at the left half of FIG. 8, although an overall lower signal was observed in conditions with 1M NaCl (as previously shown in FIG. 7), there was no loss of signal from t=1 to t=20.

Accordingly, these data suggest that the addition of salt to the sample buffer can increase sample recovery and prevent loss of viral protein on the surface of a microfluidic device.

Accordingly, in certain embodiments, a sample buffer can include salt at a concentration of at least about 0.2 M, at least about 0.3 M, or at least about 0.4 M. The salt concentration may be about 1.2 M or less, about 1.1 M or less, about 1.0 M or less, or about 0.9 M or less. Exemplary salts include chloride salts such as sodium or potassium chloride and combinations thereof.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method for detecting a coronavirus in a sample from a subject, the method comprising:
subjecting the sample to a double binding moiety sandwich assay comprising a first and a second binding moiety,
wherein the first binding moiety comprises a soluble ACE2 receptor, or a variant or fragment thereof, or an anti-coronavirus antibody,
wherein the first binding moiety is labeled with a detectable label or a capture agent, and
wherein the second binding moiety is attached to a detectable label or a capture agent, and
wherein the first binding moiety and the second binding moiety can bind the coronavirus to form a complex comprising the first binding moiety, the coronavirus, and the second binding moiety.

2. The method of claim 1, wherein at least one of the first and second binding moieties comprises a soluble ACE2 receptor, or a variant or fragment thereof.

3. The method of claim 1 or claim 2, wherein the soluble ACE2 receptor comprises an ACE2-Fc fusion protein.

4. The method of claim 3, wherein the first binding moiety comprises an anti-coronavirus antibody or fragment thereof, wherein the fragment binds to a protein of the coronavirus.

5. The method of claim 4, wherein the second binding moiety comprises an anti-coronavirus antibody or fragment thereof, wherein the fragment binds to the a protein of coronavirus.

6. The method of any one of claims 1-5, wherein the anti-coronavirus antibody or fragment thereof binds to a spike protein, a nucleocapsid protein, an envelope protein, a membrane protein, or a hemagglutinin-esterase dimer protein of a coronavirus.

7. The method of any one of claims 1-6, wherein the first binding moiety is associated, e.g., covalently associated, with the detectable label.

8. The method of any one of claims 1-6, wherein the first binding moiety is associated, e.g., covalently associated, with the capture agent.

9. The method of any one of claims 1-6 and 8, wherein the second binding moiety is associated, e.g., covalently associated, with the detectable label.

10. The method of any one of claims 1-9, wherein the second binding moiety is associated, e.g., covalently associated, with the capture agent.

11. The method of any one of claims 1-10, wherein the detectable label comprises a fluorescent label, e.g., a fluorescent latex bead.

12. The method of any one of claims 1-11, wherein the capture agent comprises a magnetic bead.

13. The method of any one of claims 1-12, wherein the method is performed within a microfluidic device.

14. The method of any one of claims 1-13, wherein the coronavirus is SARS-CoV-2.

15. The method of any one of claims 1-14, wherein the sample comprises blood, serum, or plasma.

16. The method of any one of claims 1-15, wherein the sample is contacted with a latex particle prior to subjecting the sample to the double binding moiety sandwich assay.

17. The method of any one of claims 1-16, wherein the sample is contacted with a buffer comprising a salt solution prior to subjecting the sample to the double binding moiety sandwich assay.

18. The method of any one of claims 16-17, wherein, upon subjecting the sample to the double binding moiety sandwich assay, the presence of the coronavirus is detected.

19. A microfluidic device for detecting a coronavirus in a sample from a subject, the device comprising:
a microchannel comprising a first and a second binding moiety dried within,
wherein the first binding moiety comprises a soluble ACE2 receptor, or a variant or fragment thereof, or an anti-coronavirus antibody,
wherein the first binding moiety is labeled with a detectable label or a capture agent, and
wherein the second binding moiety is attached to a detectable label or a capture agent, and
wherein the first and second binding moieties, when solubilized with the sample, form a complex comprising the first binding moiety, the coronavirus, and the second binding moiety.

20. The microfluidic device of claim 19, wherein the second binding moiety comprises a soluble ACE2 receptor, or a variant or fragment thereof.

21. The microfluidic device of claim 19 or claim 20, wherein the first and/or second binding moiety comprises an ACE2-Fc fusion protein.

22. The method of any one of claim 19-21, wherein the first binding moiety comprises an anti-coronavirus antibody or fragment thereof, wherein the fragment binds to the coronavirus.

23. The microfluidic device of any one of claim 19 or 21, wherein the second binding moiety comprises an anti-coronavirus antibody.

24. The microfluidic device of any one of claims 19-23, wherein the anti-coronavirus antibody or fragment thereof binds to a spike protein, a nucleocapsid protein, an envelope protein, a membrane protein, or a hemagglutinin-esterase dimer protein of a coronavirus.

25. The microfluidic device of any one of claims 19-24, wherein the anti-coronavirus antibody binds to a nucleocapsid protein.

26. The microfluidic device of any one of claims 19-25, wherein the first binding moiety is associated with, e.g., covalently associated, with the detectable label.

27. The microfluidic device of any one of claims 19-25, wherein the first binding moiety is associated, e.g., covalently associated, with the capture agent.

28. The microfluidic device of any one of claims 19-25 and 27, wherein the second binding moiety is associated, e.g., covalently associated, with the detectable label.

29. The microfluidic device of any one of claims 19-27, wherein the second binding moiety is associated, e.g., covalently associated, with the capture agent.

30. The microfluidic device of any one of claims 19-29, wherein the detectable label comprises a fluorescent label, e.g., a fluorescent latex bead.

31. The microfluidic device of any one of claims 19-30, wherein the capture agent comprises a magnetic bead.

32. The microfluidic device of any one of claims 19-31, wherein the coronavirus is SARS-CoV-2.

33. The microfluidic device of any one of claims 19-32, wherein the sample comprises blood, serum, or plasma.
